(19) United States
(12) Patent Application Publication
(10) Pub. No.: US 2024/0009089 A1
(43) Pub. Date: Jan. 11, 2024

(54) PEELING FORMULATION

(71) Applicant: MEDSPA S.R.L., Milano (IT)

(72) Inventor: Camilla D'ANTONIO, Milano (IT)

(21) Appl. No.: 18/255,867

(22) PCT Filed: Dec. 3, 2021
(86) PCT No.: PCT/IB2021/061298

§ 371 Date: Jun. 3, 2023

(30) Foreign Application Priority Data
Dec. 4, 2020 (IT) 102020000029813

(51) Int. Cl. A61K 8/04; A61K 8/365; A61K 8/60; A61K 8/55; A61Q 19/00

(52) U.S. Cl. CPC A61K 8/042; A61K 8/365; A61K 8/60; A61K 8/55; A61Q 19/00; A61K 2800/28

(57) ABSTRACT

The present invention relates to a cosmetic peeling formulation.

The present patent application finds application in the field of cosmetics and provides an innovative peeling formulation.

### BACKGROUND ART

Peeling is a cosmetic treatment aiming at renewing the facial skin and helping remove dark blemishes, lines and scars by means of an exfoliation mechanism.

Currently existing peeling compositions suffer from some disadvantages which limit the use thereof.

One of these is the photo sensitizing effect, whereby, following a treatment, the skin must not be exposed to sunlight so as not to cause the formation of dark blemishes on the skin.

Furthermore, many treatments are not suitable for poorly moisturized skins; therefore, they cannot be used by mature women.

Moreover, most peeling formulations are in the form of a lotion.

Therefore, there is a need for a product which does not exhibit the aforesaid disadvantages.

Prior art document US 2018/360712 describes an exfoliating composition in the form of a serum, comprising mandelic acid in a concentration of 2-10%.

The publication by Sarkar et al (Journal of Cutaneous and Aesthetic Surgery, 2019-XP055840427) describes the treatment of acne by means of a chemical peeling composition comprising 20% salicylic acid and 10% mandelic acid or phytic acid.

The product available on Mintel “Ferulic Acid Resurfacing Pads” (XP055840453) consists of swabs soaked with an exfoliating composition comprising ferulic acid, mandelic acid, lactic acid, gluconolactone, and lactobionic acid in an unknown concentration.

The exfoliating properties of lactobionic acid and gluconolactone are known from the prior art document KR 2013 0090169 (CNPSKIN Co LTD) and from the publication by Bruce Suzanne et al (J Drugs Dermatol, 2016-XP055840390), while those of mandelic acid are known from the publication by Rizza Luisa et al (Journal of Cosmetic Science, 2010) and from the publication by Edison Brenda et al (Journal of the American Academy of Dermatology, MOSBY, Inc, 2020-XP 086374028).

### SUMMARY OF THE INVENTION

The authors of the present invention have developed a peeling formulation which does not have any photosensitizing effect and offers several further advantages.

### OBJECT OF THE INVENTION

In a first object of the present invention, a cosmetic formulation for chemical peeling is described.

In a preferred aspect of the invention, such a formulation is a serum or gel-serum.

In a second object, the present invention describes the use of the peeling formulation.

In preferred aspects of the invention, such a formulation offers one or more further effects.

In a third object, the invention describes a method for a cosmetic peeling.

### DETAILED DESCRIPTION OF THE INVENTION

According to a first object, the present invention describes a peeling formulation.

Such a peeling is preferably a cosmetic peeling.

More specifically, such a formulation comprises one or more of the following ingredients: mandelic acid, lactobionic acid, gluconolactone, and phytic acid, in any qualitative and quantitative combination.

According to a preferred aspect of the present invention, the described formulation comprises all four of the above ingredients.

According to a particularly preferred aspect of the present invention, such ingredients are present in the following amounts:

| | % |
|---|---|
| ingredient | weight/weight |
| mandelic acid | 10-20% |
| lactobionic acid | 3-7% |
| gluconolactone | 2-3% |
| phytic acid | 2-3% |

According to an aspect of the present invention, the cosmetic formulation comprises:

| | % |
|---|---|
| INGREDIENT | weight/weight |
| Texture factors/rheological additives | 1.00 |
| Actives | 29.50 |
| Other excipients | 48.50 |
| (emulsifiers, preservatives, oils and | |
| fatty phases, perfumes, or essences) | |
| pH modifiers | 0.20 |
| Water | 20.80 |

where the term “active” means one or more of the compounds: mandelic acid, lactobionic acid, gluconolactone, and phytic acid.

In a preferred aspect, in a formulation of the invention such “actives” are a mixture of all four of the aforesaid ingredients.

In an even more preferred aspect, such actives comprise:

| | % |
|---|---|
| ingredient | weight/weight |
| mandelic acid | 10-20% |
| lactobionic acid | 3-7% |
| gluconolactone | 2-3% |
| phytic acid | 2-3% |

In a particular aspect of the present invention, the formulation described can comprise one or more other actives having a different property.

In a preferred aspect, the formulation described can comprise an active ingredient having a lifting action.

Such an ingredient can be acetyl-hexapeptide-8, for example.

For the purposes of the present invention, texture factors, rheological additives, emulsifiers, preservatives, oils and fatty phase components, humectants, and pH modifiers can be selected from those which are cosmetically allowed and compatible with the formulation described, as those skilled in the art would easily find.

In a particularly preferred aspect of the invention, the formulation described has the following composition:

| | % |
|---|---|
| Ingredient | weight/weight |
| Humectants | 33-61% |
| Water | 19-35% |
| Mandelic acid | 11-20% |
| Lactobionic acid | 3-8% |
| Phytic acid | 1-4% |
| Gluconolactone | 1-4% |
| Texture factors | <2% |
| pH modifiers | <1% |
| Preservative | <1% |
| Other actives | <1% |

The cosmetic formulation of the invention is preferably formulated as a serum, and more preferably as a gel-serum; this has apparent advantages since a serum formulation, and even more a gel-serum formulation, can be applied more easily, not requiring the use of dressings, cotton or special brushes, for example, allows a uniform application to the area to be treated, and stays longer thereon without dripping, thus being an easier-to-manage and safer product.

According to a second object of the invention, the use of the cosmetic peeling formulation is described.

According to preferred aspects, the use of the formulation of the invention is for one or more of the following effects: making the skin brighter and more radiant, improving the appearance of fine lines and expression lines, evening out the skin tone, making the skin firmer, moisturizing the skin, improving the appearance of blemishes, improving the skin complexion, making the skin smoother, improving the facial skin appearance.

If the formulation of the invention comprises one or more other actives, further effects will also be advantageously obtained.

For example, if an active ingredient having a lifting effect is included in the formulation of the invention, the formulation will also advantageously offer a lifting effect on the skin.

According to a third object, the present invention describes a method for a cosmetic peeling comprising the step of applying to the facial skin a suitable amount of a formulation according to the above description.

In a particular aspect, such a method comprises the step of applying a suitable amount of said formulation to the facial skin for a time of about 10 minutes and then rinsing it off.

The method should preferably be repeated three times a week.

The method can be carried out on subjects aged 18 to 60 years old.

The invention will be further described by means of the following part showing the results of the tests carried out.

Features of the Study

Test Subjects

20 women were selected, aged between 18 and 60 years old, according to the following inclusion criteria:
delicate/normal/dry skin;
good general health/absence of psychological and/or cognitive disorders
absence of dermatological and allergic diseases (cosmetic or due to other specific excipients), or other diseases (such as irritation reactions of unknown origin)
absence of drug treatments in progress which could affect the test result
non-participation in other clinical studies in the previous 30 days
obtaining informed consent

Sample Preparation

The samples were applied as a function of their features of use: to be rinsed.

Method of Applying Samples

The samples were applied 3 times a week, on alternate days, in the evening after cleansing the face and neck, as follows: massaging the product leaving a thin layer. Leaving it to take effect for 10 minutes. Rinsing off with water.

Testing

Subjective Assessments

A series of subjective assessments was gathered after a week of treatment with the product. The VNS scale was used for self-assessment, with values from 0 to 10, where 0 is the minimum value and 10 is the maximum value.

| | Value |
|---|---|
| Quality parameter | median |
| The product makes the skin brighter and | 8 |
| more radiant. | |
| The product improves the appearance of | 6 |
| fine lines and expression lines | |
| The product evens out the skin tone | 8 |
| The product makes the skin firmer | 7 |
| The product leaves the skin moisturized | 8 |
| The product improves the appearance of | 6 |
| blemishes | |
| The product improves the skin complexion | 7 |
| The product makes the skin smoother | 8 |
| The product has an immediate lifting | 7 |
| effect | |
| The product improves the facial skin | 7.5 |
| appearance | |

In particular, the effect of making the skin smoother was that with the highest average ratings: 7.8. Next, there are the effects of making the skin brighter and more radiant.

Furthermore, the results of the studies carried out showed that the formulation of the present invention is highly pleasant regardless of the subject's age and therefore is not less effective or less pleasant if applied to mature skins or skins with less moisturization.

The most important advantage among those offered by the peeling composition according to the present invention is that the negative effect of photosensitization does not occur; therefore, the product can also be advantageously used in case of more or less intense sun exposure since it does not cause skin problems.

Furthermore, the formulation provided by the present patent application is a serum or gel-serum formulation.

1. A cosmetic peeling formulation comprising mandelic acid, lactobionic acid, gluconolactone and phytic acid.

2. A cosmetic peeling formulation according to the preceding claim, comprising:

| | % |
|---|---|
| ingredient | weight/weight |
| mandelic acid | 10-20% |
| lactobionic acid | 3-7% |
| gluconolactone | 2-3% |
| phytic acid | 2-3% |

3. A cosmetic formulation according to the preceding claim 1 or 2, containing:

| | % |
|---|---|
| INGREDIENT | weight/weight |
| Texture factors/rheological additives | 1.00 |
| Actives | 29.50 |
| Other excipients | 48.50 |
| (emulsifiers, preservatives, oils and | |
| fatty phases, perfumes, or essences) | |
| pH modifiers | 0.20 |
| Water | 20.80 |

4. A cosmetic formulation according to any one of the preceding claims, having the following composition:

| | % |
|---|---|
| Ingredient | weight/weight |
| Propylene glycol | 33-61% |
| Water | 19-35% |
| Mandelic acid | 11-20% |
| Lactobionic acid | 3-8% |
| Phytic acid | 1-4% |
| Gluconolactone | 1-4% |
| Hydroxyethyl cellulose | <2% |
| Sodium hydroxide | <1% |
| Caprylic glycol | <1% |
| Acetyl hexapeptide-8 | <1% |

5. A cosmetic formulation according to any one of the preceding claims, which is in the form of a serum.

6. Use of a formulation according to any one of the preceding claims for a cosmetic peeling.

7. Use of a formulation according to any one of claims 1 to 4 for one or more of the following effects: making the skin brighter and more radiant, improving the appearance of fine lines and expression lines, evening out the skin tone, making the skin firmer, moisturizing the skin, improving the appearance of blemishes, improving the skin complexion, making the skin smoother, improving the facial skin appearance.

8. A method for a cosmetic peeling comprising the step of applying to the facial skin a suitable amount of a formulation according to any one of the preceding claims 1 to 4.

9. A peeling method according to the preceding claim, wherein a suitable amount of said formulation is applied for a time of about 10 minutes three times a week.
